(19) United States
(12) Patent Application Publication
(10) Pub. No.: US 2003/0082596 A1
(43) Pub. Date: May 1, 2003

(54) Methods of genetic analysis of probes: test3

(76) Inventor: Michael Mittmann, Palo Alto, CA (US)

Correspondence Address: Alison B. Mohr, Parsons Behle & Latimer, One Utah Center, 201 South Main Street, Suite 1800, Salt Lake City, UT, 84145-0898, US

(21) Appl. No.: 10/215,112

(22) Filed: Aug. 8, 2002

Related U.S. Application Data
(60) Provisional application No. 60/311,040, filed on Aug. 8, 2001.

(51) Int. Cl. C12Q 1/68; C12M 1/34

(52) U.S. Cl. 435/6; 435/287.2

(57) ABSTRACT

Nucleic acid sequences that are complementary, in one embodiment, to a wide variety of sequences, such as bioB, bioC, bioD, cre, dap, lys, phe, thr, trp, human actin, human GAPDH, human transferrin receptor, human transcription factor ISGF-3, human 18S rRNA, human 28S rRNA, human alu, murine actin, murine GAPDH, murine tranferrin receptor, murine pyruvate carboxylase, murine B1/B2 repetitive elements, murine 18S rRNA, rat actin, rat GAPDH, rat hexokinase, rat 5S rRNA, rat B1/B2 repetitive elements, yeast actin, yeast TATA binding factor, yeast RNA polymerase II, yeast 18S rRNA and yeast 25S RNA sequences are provided. The sequences are provided in such a way as to make them available for a variety of analyses. As such, they are related to diverse fields impacted by the nature of molecular interaction, including chemistry, biology, medicine, and medical diagnostics.

### RELATED APPLICATIONS

This application claims priority to Provisional Application Serial No. 60/311,040 filed Aug. 8, 2001, which is herein incorporated by reference in its entirety for all purposes.

### REFERENCE TO SEQUENCE LISTING

The sequence listing, including SEQ ID NOS: 1-14396, is contained on compact disc in two copies, labeled Copy 1 and Copy 2. The computer readable form is on a compact disc labeled CRF. The file name on each of the three compact discs is seqlist.rtf. The sequence listing information recorded in the computer readable form is identical to the written compact disc sequence listing. The sequence listing is hereby incorporated in this application in its entirety and is to be considered part of the disclosure of this specification.

### BACKGROUND OF THE INVENTION

The present inventions provide a unique pool of nucleic acid sequences useful for analyzing molecular interactions of biological interest. The inventions therefore relate to diverse fields impacted by the nature of molecular interaction, including chemistry, biology, medicine, and medical diagnostics.

### FIELD OF THE INVENTION

Many biological functions are carried out by regulating the expression levels of various genes, either through changes in levels of transcription (e.g. through control of initiation, provision of RNA precursors, RNA processing, etc.) of particular genes, through changes in the copy number of the genetic DNA, or through changes in protein synthesis. For example, control of the cell cycle and cell differentiation, as well as diseases, are characterized by the variations in the transcription levels of a group of genes.

Gene expression is not only responsible for physiological functions, but is also associated with pathogenesis. For example, the lack of sufficient functional tumor suppressor genes and/or the over expression of oncogene/protooncogenes leads to tumorgenesis. (See, e.g., Marshall, Cell, 64:313-326 (1991) and Weinberg, Science, 254:1138-1146 (1991)). Thus, changes in the expression levels of particular genes (e.g. oncogenes or tumor suppressors), serve as signposts for the presence and progression of various diseases.

As a consequence, novel techniques and apparatus are needed to study gene expression in specific biological systems.

All documents, i.e., publications and patent applications, cited in this disclosure, including the foregoing, are incorporated by reference herein in their entireties for all purposes to the same extent as if each of the individual documents were specifically and individually indicated to be so incorporated by reference herein in its entirety.

### SUMMARY OF THE INVENTION

The inventions provide nucleic acid sequences that are complementary to particular genes and makes them available for a variety of analyses, including, for example, gene expression analysis. For example, one embodiment the inventions comprises an array comprising of any 10 or more, 100 or more, 1000 or more, 10,000 or more or 100,000 or more nucleic acid probes containing nine or more consecutive nucleotides from the sequences listed in SEQ ID NOS: 1-14396, or the perfect match, perfect mismatch, antisense match or antisense mismatch thereof. In a further embodiment, the inventions comprise the use of any of the above arrays or fragments disclosed in SEQ ID NOS: 1-14396 to: monitor gene expression levels by hybridization of the array to a DNA library; monitor gene expression levels by hybridization to an mRNA-protein fusion compound; identify polymorphisms; identify biallelic markers; produce genetic maps; analyze genetic variation; comparatively analyze gene expression between different species; analyze gene knockouts; or to hybridize tag-labeled compounds. In a further embodiment the inventions comprises a method of analysis comprising of hybridizing one or more pools of nucleic acids to two or more of the fragments disclosed in SEQ ID NOS: 1-14396 and detecting said hybridization. In a further embodiment the inventions comprises the use of any one or more of the fragments disclosed in SEQ ID NOS: 1-14396 as a primer for polymerase chain reaction (PCR). In a further embodiment the inventions comprises the use of any one or more of the fragments disclosed in SEQ ID NOS: 1-14396 as a ligand.

### DETAILED DESCRIPTION OF THE INVENTION

I. Definitions

Massive Parallel Screening: The phrase “massively parallel screening” refers to the simultaneous screening of at least about 100, preferably about 1000, more preferably about 10,000 and more preferably about 100,000 different nucleic acid hybridizations.

Nucleic Acid: The terms “nucleic acid” or “nucleic acid molecule” refers to a deoxyribonucleotide or ribonucleotide polymer in either single-or double-stranded form, and unless otherwise limited, would encompass analogs of natural nucleotides that can function in a similar manner as naturally occurring nucleotides. Nucleic acids may be derived from a variety or sources including, but not limited to, naturally occurring nucleic acids, clones, synthesis in solution or solid phase synthesis.

Probe: As used herein a “probe” is defined as a nucleic acid capable of binding to a target nucleic acid of complementary sequence through one or more types of chemical bonds, usually through complementary base pairing, usually through hydrogen bond formation. As used herein, a probe may include natural (i.e. A, G, U, C, or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

Target nucleic acid: The term “target nucleic acid” or “target sequence” refers to a nucleic acid or nucleic acid sequence that is to be analyzed. A target can be a nucleic acid to which a probe will hybridize. The probe may or may not be specifically designed to hybridize to the target. It is either the presence or absence of the target nucleic acid that is to be detected, or the amount of the target nucleic acid that is to be quantified. The term target nucleic acid may refer to the specific subsequence of a larger nucleic acid to which the probe is directed or to the overall sequence (e.g., gene or mRNA) whose expression level it is desired to detect. The difference in usage will be apparent from context.

mRNA or transcript: The term “mRNA” refers to transcripts of a gene. Transcripts are RNA including, for example, mature messenger RNA ready for transaction, products of various stages of transcript processing. Transcript processing may include splicing, editing and degradation.

Subsequence: “Subsequence” refers to a sequence of nucleic acids that comprise a part of a longer sequence of nucleic acids.

Perfect match: The term “match,” “perfect match,” “perfect match probe” or “perfect match control” refers to a nucleic acid that has a sequence that is designed to be perfectly complementary to a particular target sequence. The nucleic acid is typically perfectly complementary to a portion (subsequence) of the target sequence. A perfect match (PM) probe can be a “test probe”, a “normalization control” probe, an expression level control probe and the like. A perfect match control or perfect match is, however, distinguished from a “mismatch” or “mismatch probe.”

Mismatch: The term “mismatch,” “mismatch control” or “mismatch probe” refers to a nucleic acid whose sequence is deliberately selected not to be perfectly complementary to a particular target sequence. As a non-limiting example, for each mismatch (MM) control in a high-density probe array there typically exists a corresponding perfect match (PM) probe that is perfectly complementary to the same particular target sequence. The mismatch may comprise one or more bases. While the mismatch(es) may be located anywhere in the mismatch probe, terminal mismatches are less desirable because a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions. A homo-mismatch substitutes an adenine (A) for a thymine (T) and vice versa and a guanine (G) for a cytosine (C) and vice versa. For example, if the target sequence was AGGTCCA, a probe designed with a single homo-mismatch at the central, or fourth position, would result in the following sequence: TCCTGGT.

Array: An “array” is a solid support with at least a first surface having a plurality of different nucleic acid sequences attached to the first surface.

Gene Knockout: the term “gene knockout,” as defined in Lodish et al. Molecular Cell Biology 3^{rd} Edition, Scientific American Books pub., which is hereby incorporated in its entirety for all purposes is, is a technique for selectively inactivating a gene by replacing it with a mutant allele in an otherwise normal organism.

DNA Library: as used herein the term “genomic library” or “genomic DNA library” refers to a collection of cloned DNA molecules consisting of fragments of the entire genome (genomic library) or of DNA copies of all the mRNA produced by a cell type (cDNA library) inserted into a suitable cloning vector.

Polymorphism: a “polymorphism” refers to the occurrence of two or more genetically determined alternative sequences or alleles in a population. A polymorphic marker or site is the locus at which divergence occurs. Preferred markers have at least two alleles, each occurring at a frequency of greater than 1%, and more preferably greater than 10% or 20% of the selected population. A polymorphic locus may be as small as one base pair. Polymorphic markers include restriction fragment length polymorphisms, variable number or tandem repeats (VNTR's), hypervariable regions, minisatellites, dinucleotide repeats, trinucleotide repeats, tetranucleotide repeats, simple sequence repeats, and insertion elements such as ALU. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic or biallelic polymorphism has two forms. A triallelic polymorphism has three forms.

Genetic map: a map which presents the order of specific sequences on a chromosome.

Genetic variation: refers to variation in the sequence of the same region between two or more organisms.

Hybridization: the association of two complementary nucleic acid strands or their derivatives (such as PNA) to form double stranded molecules. Hybrids can contain two DNA strands, two RNA strands, or one DNA and one RNA strand.

mRNA-protein fusion: a compound whereby an mRNA is directly attached to the peptide or protein it incodes by a stable covalent linkage.

Ligand: any molecule, other than an enzyme substrate, that binds tightly and specifically to a macromolecule, for example, a protein, forming a macromolecule-ligand complex.

II. General

SEQ ID NOS: 1-14396, encompassed in the Sequence Listing, presents target sequences included in the inventions. Each target sequence corresponds to and represents at least four additional nucleic acid sequences included in the inventions. For example, the first nucleic acid sequence listed in SEQ ID NOS: 1-14396 is: 5′-aggacttgccatcgtagaactgctg-3′ the additional sequences included in the inventions that are represented by this nucleic acid sequence are, for example:

3′-tcctgaacggtagcatcttgacgac-5′ = (perfect) sense
match
3′-tcctgaacggtaccatcttgacgac-5′ = sense mismatch
3′-aggacttgccatcgtagaactgctg-5′ = (perfect) anti-
sense match
3′-aggacttgccatggtagaactgctg-5′ = antisense mis-
match

Accordingly, for each nucleic acid sequence listed in SEQ ID NOS: 1-14396, this disclosure includes the corresponding sense match, sense mismatch, antisense match and antisense mismatch. The position of the mismatch is not limited to the above example, it may be located anywhere in the nucleic acid sequence and may comprise one or more bases.

Consequently, the present inventions includes: a) the target sequences listed in SEQ ID NOS: 1-14396, or the sense match, sense mismatch, antisense match or antisense mismatch thereof; b) clones that comprise the target nucleic acid sequences listed in SEQ ID NOS: 1-14396, or the sense match, sense mismatch, antisense match or antisense mismatch thereof; c) longer nucleotide sequences that include the nucleic acid sequences listed in SEQ ID NOS: 1-14396, or the sense match, sense mismatch, antisense match or antisense mismatch thereof and d) subsequences greater than nine nucleotides in length of the target nucleic acid sequences listed in SEQ ID NOS: 1-14396, or the sense match, sense mismatch, antisense match or antisense mismatch.

Target sequences were chosen from clusters of known sequences, such as bioB, bioC, bioD, cre, dap, lys, phe, thr, trp, human actin, human GAPDH, human transferrin receptor, human transcription factor ISGF-3, human 18S rRNA, human 28S rRNA, human alu, murine actin, murine GAPDH, murine tranferrin receptor, murine pyruvate carboxylase, murine B1/B2 repetitive elements, murine 18S rRNA, rat actin, rat GAPDH, rat hexokinase, rat 5S rRNA, rat B1/B2 repetitive elements, yeast actin, yeast TATA binding factor, yeast RNA polymerase II, yeast 18S rRNA and yeast 25S RNA sequences. Target sequences were selected using the computer based methods described in U.S. Pat. No. 6,309,822 incorporated herein by reference for all purposes.

The present inventions provide a pool of unique nucleotide sequences complementary to sequences, such as bioB, bioC, bioD, cre, dap, lys, phe, thr, trp, human actin, human GAPDH, human transferrin receptor, human transcription factor ISGF-3, human 18S rRNA, human 28S rRNA, human alu, murine actin, murine GAPDH, murine tranferrin receptor, murine pyruvate carboxylase, murine B1/B2 repetitive elements, murine 18S rRNA, rat actin, rat GAPDH, rat hexokinase, rat 5S rRNA, rat B1/B2 repetitive elements, yeast actin, yeast TATA binding factor, yeast RNA polymerase II, yeast 18S rRNA and yeast 25S RNA sequences in particular embodiments which alone, or in combinations of two or more, 10 or more, 100 or more, 1,000 or more, 10,000 or more, or 100,000 or more, can be used for a variety of applications.

In one embodiment, the present inventions provide for a pool of unique nucleotide sequences which are complementary to approximately 14396 sequences, such as bioB, bioC, bioD, cre, dap, lys, phe, thr, trp, human actin, human GAPDH, human transferrin receptor, human transcription factor ISGF-3, human 18S rRNA, human 28S rRNA, human alu, murine actin, murine GAPDH, murine tranferrin receptor, murine pyruvate carboxylase, murine B1/B2 repetitive elements, murine 18S rRNA, rat actin, rat GAPDH, rat hexokinase, rat 5S rRNA, rat B1/B2 repetitive elements, yeast actin, yeast TATA binding factor, yeast RNA polymerase II, yeast 18S rRNA and yeast 25S RNA sequences formed into a high density array of probes suitable for array based massive parallel gene expression. Array based methods for monitoring gene expression are disclosed and discussed in detail in U.S. Pat. No. 5,800,992, U.S. Pat. No. 6,309,822, and PCT Application WO 92/10588 (published on Jun. 25, 1992), all of which are incorporated herein by reference for all purposes. Generally those methods of monitoring gene expression involve (1) providing a pool of target nucleic acids comprising RNA transcript(s) of one or more target gene(s), or nucleic acids derived from the RNA transcript(s); (2) hybridizing the nucleic acid sample to a high density array of probes and (3) detecting the hybridized nucleic acids and calculating a relative expression (transcription, RNA processing or degradation) level.

The development of Very Large Scale Immobilized Polymer Synthesis or VLSIPS™ technology has provided methods for making very large arrays of nucleic acid probes in very small arrays. See U.S. Pat. No. 5,143,854 and PCT Patent Publication Nos. WO 90/15070 and WO 92/10092, and Fodor et al., Science, 251, 767-77 (1991), each of which is incorporated herein by reference. U.S. patent application Ser. No. 08/670,118, describes methods for making arrays of nucleic acid probes that can be used to detect the presence of a nucleic acid containing a specific nucleotide sequence. Methods of forming high density arrays of nucleic acids, peptides and other polymer sequences with a minimal number of synthetic steps are known. The nucleic acid array can be synthesized on a solid substrate by a variety of methods, including, but not limited to, light-directed chemical coupling and mechanically directed coupling. In a preferred detection method, the array of immobilized nucleic acids, or probes, is contacted with a sample containing target nucleic acids, to which a flourescent label is attached. Target nucleic acids hybridize to the probes on the array and any nonhybridized nucleic acids are removed. The array containing the hybridized target nucleic acids are exposed to light which excites the flourescent label. The resulting flourescent intensity, or brightness, is detected. Relative brightness is used to determine which probe is the best candidate for the perfect match to the hybridized target nucleic acid because flourescent intensity (brightness) corresponds to binding affinity. Once the position of the perfect match probe is known, the sequence of the hybridized target nucleic is known because the sequence and position of the probe is known.

In the array of the present inventions the probes are presented in pairs, one probe in each pair being a perfect match to the target sequence and the other probe being identical to the perfect match probe except that the central base is a homo-mismatch. Mismatch probes provide a control for non-specific binding or cross-hybridization to a nucleic acid in the sample other than the target to which the probe is directed. Thus, mismatch probes indicate whether a hybridization is or is not specific. For example, if the target is present, the perfect match probes should be consistently brighter than the mismatch probes because fluorescence intensity, or brightness, corresponds to binding affinity. (See, for example U.S. Pat. No. 5,324,633, which is incorporated herein for all purposes.) In addition, if all central mismatches are present, the mismatch probes can be used to detect a mutation. Finally the difference in intensity between the perfect match and the mismatch probe (I(PM)-I(MM)) provides a good measure of the concentration of the hybridized material. See pending PCT Application No. 98/11223, which is incorporated herein by reference for all purposes. The probe pairs are presented in both sense and antisense orientation, thereby eliciting a total of four probes per target sequence: sense match, sense mismatch, antisense match and antisense mismatch.

In a preferred embodiment, an array comprising probes corresponding to SEQ ID NOS: 1-14396 or the perfect match, perfect mismatch, antisense match, and antisense mismatch thereof may be used to monitor experimental parameters and/or sample preparation prior to engaging in experiments with larger, more expensive arrays. The sequences in SEQ ID NOS: 1-14396 correspond to a wide variety of organisms and thus can be used to aid in optimization and monitoring of experimental parameters and the sample preparation process.

In another embodiment, the current inventions provide a pool of sequences that may be used as probes for their complementary genes listed in the Genbank database. Methods for making probes are well known. See for example Sambrook, Fritsche and Maniatis. “Molecular Cloning A laboratory Manual” 2^{nd} Ed. Cold Spring Harbor Press (1989) (“Maniatis et al.”) which is hereby incorporated in its entirety by reference for all purposes. Maniatis et al. describes a number of uses for nucleic acid probes of defined sequence. Some of the uses described by Maniatis et al. include: to screen cDNA or genomic DNA libraries, or subclones derived from them, for additional clones containing segments of DNA that have been isolated and previously sequenced; in Southern, northern, or dot-blot hybridization to identify or detect the sequences of specific genes; in Southern, or dot-blot hybridization of genomic DNA to detect specific mutations in genes of known sequence; to detect specific mutations generated by site-directed mutagenesis of cloned genes; and to map the 5′ termini of mRNA molecules by primer extensions. Maniatis et al. describes other uses for probes throughout. See also Alberts et al. Molecular Biology of the Cell 3^{rd} edition, Garland Publishing Inc. (1994) p. 307 and Lodish et al. Molecular Cell Biology, 3^{rd} edition, Scientific American Books (1995) p. 285-286, each of which is hereby incorporated by reference in its entirety for all purposes, for a brief discussion of the use of nucleic acid probes in in situ hybridization. Other uses for probes derived from the sequences disclosed in the present inventions will be readily apparent to those of skill in the art. See, for example, Lodish et al. Molecular Cell Biology, 3^{rd} edition, Scientific American Books (1995) p.229-233, incorporated above, for a description of the construction of genomic libraries.

In another embodiment, the current inventions may be combined with known methods to monitor expression levels of genes in a wide variety of contexts. For example, where the effects of a drug on gene expression are to be determined, the drug will be administered to an organism, a tissue sample, or a cell and the gene expression levels will be analyzed. For example, nucleic acids are isolated from the treated tissue sample, cell, or a biological sample from the organism and from an untreated organism tissue sample or cell, hybridized to a high density probe array containing probes directed to the gene of interest and the expression levels of that gene are determined. The types of drugs that may be used in these types of experiments include, but are not limited to, antibiotics, antivirals, narcotics, anti-cancer drugs, tumor suppressing drugs, and any chemical composition that may affect the expression of genes in vivo or in vitro. The current inventions are particularly suited to be used in the types of analyses described by, for example, pending U.S. Pat. No. 6,309,822 and PCT Application No. 98/11223, each of which is incorporated by reference in its entirety for all purposes. As described in Wodicka et al., Nature Biotechnology 15 (1997), hereby incorporated by reference in its entirety for all purposes, because mRNA hybridization correlates to gene expression level, hybridization patterns can be compared to determine differential gene expression. As non-limiting examples: hybridization patterns from samples treated with certain types of drugs may be compared to hybridization patterns from samples that have not been treated or that have been treated with a different drug; hybridization patterns for samples infected with a specific virus may be compared against hybridization patterns from non-infected samples; hybridization patterns for samples with cancer may be compared against hybridization patterns for samples without cancer; hybridization patterns of samples from cancerous cells which have been treated with a tumor suppressing drug may be compared against untreated cancerous cells, etc. Zhang et al., Science 276 1268-1272, hereby incorporated by reference in its entirety for all purposes, provides an example of how gene expression data can provide a great deal of insight into cancer research. One skilled in the art will appreciate that a wide range of applications will be available using two or more, 10 or more, 100 or more, 1000 or more, 10,000 or more or 100,000 or more of the SEQ ID NOS: 1-14396 sequences as probes for gene expression analysis. The combination of the DNA array technology and sequences such as the bioB, bioC, bioD, cre, dap, lys, phe, thr, trp, human actin, human GAPDH, human transferrin receptor, human transcription factor ISGF-3, human 18S rRNA, human 28S rRNA, human alu, murine actin, murine GAPDH, murine tranferrin receptor, murine pyruvate carboxylase, murine B1/B2 repetitive elements, murine 18S rRNA, rat actin, rat GAPDH, rat hexokinase, rat 5S rRNA, rat B1/B2 repetitive elements, yeast actin, yeast TATA binding factor, yeast RNA polymerase II, yeast 18S rRNA and yeast 25S RNA sequence specific probes in this disclosure is a powerful tool for studying gene expression.

In another embodiment, the inventions may be used in conjunction with the techniques which link specific proteins to the mRNA which encodes the protein. See for example, Roberts and Szostak Proc. Natl. Acad. Sci. 94 12297-12302 (1997), which is incorporated herein in its entirety for all purposes. Hybridization of these mRNA protein fusion compounds to arrays comprised of two or more, 10 or more, 100 or more, 1000 or more, 10,000 or more, or 100,000 or more the sequences disclosed in the present inventions provides a powerful tool for monitoring expression levels.

In one embodiment, the current inventions provide a pool of unique nucleic acid sequences that can be used for parallel analysis of gene expression under selective conditions. Without wishing to be limited, genetic selection under selective conditions could include: variation in the temperature of the organism's environment; variation in pH levels in the organism's environment; variation in an organism's food (type, texture, amount etc.); variation in an organism's surroundings; etc. Arrays, such as those in the present inventions, can be used to determine whether gene expression is altered when an organism is exposed to selective conditions.

Methods for using nucleic acid arrays to analyze genetic selections under selective conditions are known. See for example, R. Cho et al., Proc. Natl. Acad. Sci. 95 3752-3757 (1998), incorporated herein in its entirety for all purposes. Cho et al. describes the use of a high-density array containing oligonucleotides complementary to every gene in the yeast Saccharomyces cerevisiae to perform two-hybrid protein-protein interaction screens for S. cerevisiae genes implicated in mRNA splicing and microtubule assembly. Cho et al. was able to characterize the results of a screen in a single experiment by hybridization of labeled DNA derived from positive clones. Briefly, as described by Cho et al., two proteins are expressed in yeast as fusions to either the DNA-binding domain or the activation domain of a transcription factor. Physical interaction of the two proteins reconstitutes transcriptional activity, turning on a gene essential for survival under selective conditions. In screening for novel protein-protein interactions, yeast cells are first transformed with a plasmid encoding a specific DNA-binding fusion protein. A plasmid library of activation domain fusions derived from genomic DNA is then introduced into these cells. Transcriptional activation fusions found in cells that survive selective conditions are considered to encode peptide domains that may interact with the DNA-binding domain fusion protein. Clones are then isolated from the two-hybrid screen and mixed into a single pool. Plasmid DNA is purified from the pooled clones and the gene inserts are amplified using PCR. The DNA products are then hybridized to yeast whole genome arrays for characterization. The methods employed by Cho et al. are applicable to the analysis of a range of genetic selections. High density arrays created using two or more, 10 or more, 100 or more, 1000 or more, 10,000 or more, or 100,000 or more of the sequences disclosed in the current inventions can be used to analyze genetic selections in the human, rat, murine or yeast system using the methods described in Cho et al.

In another embodiment, the current inventions provide a pool of unique nucleic acid sequences that can be used to identify biallelic markers, providing a novel and efficient approach to the study of genetic variation. For example, methods for using high density arrays comprised of probes that are complementary to the genomic DNA of a particular species to interrogate polymorphisms are well known. See for example, U.S. patent application Ser. No. 08/965,620 and U.S. Pat. No. 6,300,063, which are hereby incorporated herein for all purposes. Pools of two or more, 10 or more, 100 or more, 1000 or more, 10,000 or more, or 100,000 or more of the sequences disclosed in this invention combined with the methods described in the above patent applications provides a tool for studying genetic variation in the human, rat, murine, or yeast system.

In another embodiment of the inventions, genetic variation can be used to produce genetic maps of various strains of different species. Winzeler et al., “Direct Allelic Variation Scanning of the Yeast Genome” Science. 281(5380):1194-7. (1998), which is hereby incorporated for all purposes describe methods for conducting this type of screening with arrays containing probes complementary to the yeast genome. Briefly, genomic DNA from strains that are phenotypically different are isolated, fragmented, and labelled. Each strain is then hybridized to identical arrays comprised of the nucleic acid sequences complementary to the system being studied. Comparison of hybridization patterns between the various strains then serve as genetic markers. As described by Winzler et al, these markers can then be used for linkage analysis. High density arrays created from two or more, 10 or more, 100 or more, 1000 or more, 10,000 or more, or 100,000 or more of the sequences disclosed in the inventions can be used to study genetic variation using the methods described by Winzler et al.

In another embodiment, the present inventions may be used for cross-species comparisons. One skilled in the art will appreciate that it is often useful to determine whether a gene present in one species, for example the rat, is present in a conserved format in another species, including, without limitation, mouse, human, chicken, zebrafish, drosophila, or yeast. See, for example, Andersson et al., Mamm Genome 7(10):717-734 (1996), which is hereby incorporated by reference for all purposes, which describes the utility of cross-species comparisons. The use of two or more, 10 or more, 100 or more, 1000 or more, 10,000 or more or 100,000 or more of the sequences disclosed in the inventions in an array can be used to determine whether any of the sequence from one or more of the human, rat, mouse or yeast genes represented by the sequences disclosed in the inventions are conserved in another species by, for example, hybridizing genomic nucleic acid samples from another species to an array comprised of the sequences disclosed in the inventions. Areas of hybridization will yield genomic regions where the nucleotide sequence is highly conserved between the interrogation species and sequences such as the bioB, bioC, bioD, cre, dap, lys, phe, thr, tip, human actin, human GAPDH, human transferrin receptor, human transcription factor ISGF-3, human 18S rRNA, human 28S rRNA, human alu, murine actin, murine GAPDH, murine tranferrin receptor, murine pyruvate carboxylase, murine B1/B2 repetitive elements, murine 18S rRNA, rat actin, rat GAPDH, rat hexokinase, rat 5S rRNA, rat B1/B2 repetitive elements, yeast actin, yeast TATA binding factor, yeast RNA polymerase II, yeast 18S rRNA and yeast 25S RNA sequences disclosed in the inventions.

In another embodiment, the present inventions may be used to characterize the genotype of knockouts. Methods for using gene knockouts to identify a gene are well known. See for example, Lodish et al. Molecular Cell Biology, 3^{rd} Edition, Scientific American Books pub pp. 292-296 and U.S. Pat. No. 5,679,523, which are hereby incorporated by reference for all purposes. By isolating genomic nucleic acid samples from knockout species with a known phenotype and hybridizing the samples to an array comprised of two or more, 10 or more, 100 or more, 1000 or more, 10,000 or more, or 100,000 or more of the sequences disclosed in the inventions, candidates genes which contribute to the phenotype will be identified and made accessible for further characterization.

In another embodiment, the present inventions may be used to identify new gene family members. Methods of screening libraries with probes are well known. (See, for example, Maniatis et al, incorporated by reference above.) Because the present inventions are comprised of nucleic acid sequences from specific known genes, two or more, 10 or more, 100 or more, 1000 or more, 10,000 or more, or 100,000 or more of sequences disclosed in the inventions may be used as probes to screen genomic libraries to look for additional family members of those genes from which the target sequences are derived. In another embodiment, the present inventions may be used to provide nucleic acid sequences to be used as tag sequences. Tag sequences are a type of genetic “bar code” that can be used to label compounds of interest. The analysis of deletion mutants using tag sequences is described in, for example, Shoemaker et al., Nature Genetics 14 450-456 (1996), which is hereby incorporated by reference in its entirety for all purposes. Shoemaker et al. describes the use of PCR to generate large numbers of deletion strains. Each deletion strain is labelled with a unique 20-base tag sequence that can be hybridized to a high-density oligonucleotide array. The tags serve as unique identifiers (molecular bar codes) that allow analysis of large numbers of deletion strains simultanously through selective growth conditions. The use of tag sequences need not be limited to this example however. The utility of using unique known short oligonucleotide sequences capable of hybridizing to a nucleic acid array to label various compounds will be apparent to one skilled in the art. One or more, 10 or more, 100 or more, 1000 or more, 10,000 or more, or 100,000 or more of the SEQ ID NOS: 1-14396 sequences are excellent candidates to be used as tag sequences.

In another embodiment of the inventions, the sequences of the inventions may be used to generate primers directed to their corresponding genes as disclosed in the Genbank or any other public database. These primers may be used in such basic techniques as sequencing or PCR, see for example Maniatis et al., incorporated by reference above.

In another embodiment, the inventions provide a pool of nucleic acid sequences to be used as ligands for specific genes. The sequences disclosed in the inventions may be used as ligands to their corresponding genes as disclosed in the Genbank or any other public database. Compounds that specifically bind known genes are of interest for a variety of uses. One particular clinical use is to act as an antisense protein that specifically binds and disables a gene that has been, for example, linked to a disease. Methods and uses for ligands to specific genes are known. See for example, U.S. Pat. No. 5,723,594, which is hereby incorporated by reference in its entirety for all purposes. In a preferred embodiment, the hybridized nucleic acids are detected by detecting one or more labels attached to the sample nucleic acids. The labels may be incorporated by any of a number of means well known to those of skill in the art. In one embodiment, the label is simultaneously incorporated during the amplification step in the preparation of the sample nucleic acids. Thus, for example, polymerase chain reaction with labeled primers or labeled nucleotides will provide a labeled amplification product. In a another embodiment, transcription amplification, as described above, using a labeled nucleotide (e.g. fluorescein-labeled UTP and/or CTP) incorporates a label into the transcribed nucleic acids.

Alternatively, a label may be added directly to the original nucleic acid sample (e.g., mRNA, polyA mRNA, cDNA, etc.) or to the amplification product after the amplification is completed. Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore). Detectable labels suitable for use in the present inventions include any composition detectable by spectroscopic, photochemical, biochemical, immunochemical, electrical, optical or chemical means. Useful labels in the present inventions include biotin for staining with labeled streptavidin conjugate, magnetic beads (e.g., Dynabead S), fluorescent dyes (e.g., fluorescein, texas red, rhodamine, green fluorescent protein, and the like), radiolabels (e.g., 3H, 125₁, 35_{S,} 14C, or ³²p), phosphorescent labels, enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241, each of which is hereby incorporated by reference in its entirety for all purposes.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The label may be added to the target nucleic acid(s) prior to, or after the hybridization. So called “direct labels” are detectable labels that are directly attached to or incorporated into the target nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an aviden-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993), which is hereby incorporated by reference in its entirety for all purposes.

Fluorescent labels are preferred and easily added during an in vitro transcription reaction. In a preferred embodiment, fluorescein labeled UTP and CTP are incorporated into the RNA produced in an in vitro transcription reaction as described above.

### EXAMPLE

The following example serves to illustrate the type of experiment that could be conducted using the inventions.

Expression Monitoring by Hybridization to High Density Oligonucleotide Arrays

Arrays containing the desired number of probes can be synthesized using the method described in U.S. Pat. No. 5,143,854, incorporated by reference above. Extracted poly (A)⁺RNA can then be coverted to cDNA using the methods described below. The cDNA is then transcribed in the presence of labeled ribonucleotide triphosphates. The label may be biotin or a dye such as fluorescein. RNA is then fragmented with heat in the presence of magnesium ions. Hybridizations are carried out in a flow cell that contains the two-dimensional DNA probe arrays. Following a brief washing step to remove unhybridized RNA, the arrays are scanned using a scanning confocal microscope.

1. A Method of RNA Preparation

Labeled RNA is prepared from clones containing a T7 RNA polymerase promoter site by incorporating labeled ribonucleotides in an IVT reaction. Either biotin-labeled or fluorescein-labeled UTP and CTP (1:3 labeled to unlabeled) plus unlabeled ATP and GTP is used for the reaction with 2500 U of T7 RNA polymerase. Following the reaction unincorporated nucleotide triphosphates are removed using size-selective membrane such as Microcon-100, (Amicon, Beverly, Mass.). The total molar concentration of RNA is based on a measurement of the absorbance at 260 rim. Following quantitation of RNA amounts, RNA is fragmented randomly to an average length of approximately 50 bases by heating at 940 in 40 mM Tris-acetate pH 8.1, 100 mM potassium acetate, 30 MM magnesium acetate, for 30 to 40 minutes. Fragmentation reduces possible interference from RNA secondary structure, and minimizes the effects of multiple interactions with closely spaced probe molecules. For material made directly from cellular RNA, cytoplasmic RNA is extracted from cells by the method of Favaloro et al. Methods Enzymol. 65:718-749 (1980) hereby incorporated by reference for all purposes, and poly (A)⁺RNA is isolated with an oligo dT selection step using, for example, Poly Atract, (Promega, Madison, Wis.). RNA can be amplified using a modification of the procedure described by Eberwine et al. Proc. Natl. Acad. Sci. USA 89:3010-3014 (1992), hereby incorporated by reference for all purposes. Microgram amounts of poly (A)⁺RNA are converted into double stranded cDNA using a cDNA synthesis kit (kits may be obtained from Life Technologies, Gaithersburg, Md.) with an oligo dT primer incorporating a T7 RNA polymerase promoter site. After second-strand synthesis, the reaction mixture is extracted with phenol/chloroform, and the double-stranded DNA isolated using a membrane filtration step using, for example, Microcon-100, (Amicon). Labeled cRNA can be made directly from the cDNA pool with an IVT step as described above. The total molar concentration of labeled cRNA is determined from the absorbance at 260 nm and assuming an average RNA size of 1000 ribonucleotides. The commonly used convention is that 1 OD is equivalent to 40 ug of RNA, and that 1 ug of cellular mRNA consists of 3 pmol of RNA molecules. Cellular mRNA may also be labeled directly without any intermediate cDNA synthesis steps. In this case, Poly (A)⁺RNA is fragmented as described, and the 5′ ends of the fragments are kinased and then incubated overnight with a biotinylated oligoribonucleotide (5′-biotin-AAAAAA-3′) in the presence of T4 RNA ligase (available from Epicentre Technologies, Madison, Wis.). Alternatively, mRNA has been labeled directly by UV-induced cross-linking to a psoralen derivative linked to biotin (available from Schleicher & Schuell, Keene, N.H.).

2. Array Hybridization and Scanning:

Array hybridization solutions can be made containing 0.9 M NaCl, 60 mM EDTA, and 0.005% Triton X-100, adjusted to pH 7.6 (referred to as 6×SSPE-T). In addition, the solutions should contain 0.5 mg/ml unlabeled, degraded herring sperm DNA (available from Sigma, St. Louis, Mo.). Prior to hybridization, RNA samples are heated in the hybridization solution to 99° C. for 10 minutes, placed on ice for five minutes, and allowed to equilibrate at room temperature before being placed in the hybridization flow cell. Following hybridization, the solutions are removed, the arrays washed with 6×SSPE-T at 22 C. for seven minutes, and then washed with 0.5×SSPE-T at 40° C. for 15 minutes. When biotin labeled RNA is used the hybridized RNA should be stained with a streptavidinphycoerythrin in 6×SSPE-T at 40° C. for five minutes. The arrays are read using a scanning confocal microscope made by Molecular Dynamics (commercially available through Affymetrix, Santa Clara, Calif.). The scanner uses an argon ion laser as the excitation source, with the emission detected by a photomultiplier tube through either a 530 nm bandpass filter (flourescein) or a 560 nm longpass filter (phycoerythrin). Nucleic acids of either sense or antisense orientations may be used in hybridization experiments. Arrays for probes with either orientation (reverse complements of each other) are made using the same set of photolithgraphic masks by reversing the order of the photochemical steps and incorporating the complementary nucleotide.

3. Quantitative Analysis of Hybridization Patterns and Intensities

Following a quantitative scan of an array, a grid is aligned to the image using the known dimensions of the array and the corner control regions as markers. The image is then reduced to a simple text file containing position and intensity information using software developed at Affymetrix (available with the confocal scanner). This information is merged with another text file that contains information relating physical position on the array to probe sequence and the identity of the RNA (and the specific part of the RNA) for which the oligonucleotide probe is designed. The quantitative analysis of the hybridization results involves a simple form of pattern recognition based on the assumption that, in the presence of a specific RNA, the perfect match (PM) probes will hybridize more strongly on average than their mismatch (MM) partners. The number of instances in which the PM hybridization is larger than the MM signal is computed along with the average of the logarithm of the PM/MM ratios for each probe set. These values are used to make a decision (using a predefined decision matrix) concerning the presence or absence of an RNA. To determine the quantitative RNA abundance, the average of the difference (PM-MM) for each probe family is calculated. The advantage of the difference method is that signals from random cross-hybridization contribute equally, on average, to the PM and MM probes, while specific hybridization contributes more to the PM probes. By averaging the pairwise differences, the real signals add constructively while the contributions from cross-hybridization tend to cancel. When assessing the differences between two different RNA samples, the hybridization signals from side-by-side experiments on identically synthesized arrays are compared directly. The magnitude of the changes in the average of the difference (PM-MM) values is interpreted by comparison with the results of spiking experiments as well as the signals observed for the internal standard bacterial and phase RNAs spiked into each sample at a known amount. Data analysis programs, such as those described in U.S. patent application Ser. No. 08/828,952 perform these operations automatically.

### CONCLUSION

The inventions herein provide a pool of unique nucleic acid sequences that are complementary to approximately 8124 specific known sequences, such as bioB, bioC, bioD, cre, dap, lys, phe, thr, trp, human actin, human GAPDH, human transferring receptor, human transcription factor ISGF-3, human 18S rRNA, human 28S rRNA, human alu, murine actin, murine GAPDH, murine tranferrin receptor, murine pyruvate carboxylase, murine B1/B2 repetitive elements, murine 18S rRNA, rat actin, rat GAPDH, rat hexokinase, rat 5S rRNA, rat, B1/B2 repetitive elements, yeast actin, yeast TATA binding factor, yeast RNA polymerase II, yeast 18S rRNA and yeast 25S RNA sequences. These sequences can be used for a variety of types of analyses.

The above description is illustrative and not restrictive. Many variations of the inventions will become apparent to those of skill in the art upon review of this disclosure. The scope of the inventions should, therefore, be determined not with reference to the above description, but instead be determined with reference to the appended claims along with their full scope of equivalents.

What is claimed is:

1. An array comprising at least 10 distinct nucleic acid probes, each probe being comprised of 9 or more consecutive nucleotides from at least one of the sequences listed in SEQ ID NOS: 1-14396, or the perfect sense match, sense mismatch, antisense match or antisense mismatch thereof.

2. The array of claim 1 comprising a plurality of nucleic acid probes, wherein each probe comprises one of the sequences listed in SEQ ID NOS: 1-14396 or the perfect sense match, sense mismatch, antisense match or antisense mismatch thereof.

3. The array of claim 1 wherein said array is used to monitor gene expression levels by hybridization to a DNA library.

4. The array of claim 1 wherein said array is used for analysis of genetic variation.

5. The array of claim 1 wherein said array is used for hybridization of tag-labeled compounds.

6. A method of analysis comprising:
a) hybridizing one or more nucleic acids to two or more nucleic acid probes, each probe comprising at least one of the sequences listed in SEQ ID NOS: 1-14396, or the perfect sense match, sense mismatch, antisense match or antisense mismatch thereof; and
b) detecting said hybridization.

7. The method of claim 6 wherein said nucleic acid probes are attached to a solid support.

8. The method of claim 6 wherein said analysis comprises monitoring gene expression levels.

9. The method of claim 8 wherein said monitoring gene expression levels comprises comparing gene expression levels of nucleic acids derived from two or more different samples and further comprises the step of: comparing said hybridization patterns between said nucleic acids derived from said two or more different samples.

10. The method of claim 6 wherein said method of analysis comprises identifying biallelic markers.

11. The method of claim 6 wherein said method of analysis comprises identifying polymorphisms.

12. The method of claim 6 wherein said method of analysis comprises a crossspecies comparison wherein the hybridization patterns of a pool of nucleic acids derived from one species are compared with the hybridization patterns of a pool of nucleic acids derived from another species.

13. The method of claim 6 wherein each of said nucleic acids further comprise a tag sequence.

14. The method of claim 6 wherein said method of analysis is a method of identifying family members of a gene.

15. A method comprising using any one or more nucleic acid sequences comprising at least one of the sequences listed in SEQ ID NOS: 1-14396, or the perfect sense match, sense mismatch, antisense match or antisense mismatch thereof as a probe.

16. The method of claim 15 wherein said probe is used in an in situ hybridization.

17. The method of claim 15 wherein said probe is used to screen cDNA or genomic libraries, or subclones derived from them, for additional clones containing segments of DNA that have been isolated and previously sequenced.

18. The method of claim 15 wherein said probe is used in Southern, northern, or dot-blot hybridization to identify or detect the sequence of any gene.

19. The method of claim 15 wherein said probe is used in Southern or dot-blot hybridization of genomic DNA to detect specific mutations in any gene.

20. The method of claim 15 wherein said probe is used to map the 5′ termini of mRNA molecules by primer extensions.
